(19) United States
(12) Patent Application Publication
(10) Pub. No.: US 2025/0032373 A1
(43) Pub. Date: Jan. 30, 2025

(54) A PERSONAL CARE COMPOSITION

(71) Applicant: Conopco Inc., d/b/a UNILEVER, Englewood Cliffs, NJ (US)

(72) Inventors: Guoqiang CHEN, Shanghai (CN); Leilei LYU, Shanghai (CN); Xiaoli WANG, Shanghai (CN); Yun WANG, Shanghai (CN); Xiaoxia YANG, Shanghai (CN); Shangchun YI, Shanghai (CN)

(73) Assignee: Conopco Inc., d/b/a UNILEVER, Englewood Cliffs, NJ (US)

(21) Appl. No.: 18/714,810

(22) PCT Filed: Dec. 5, 2022
(86) PCT No.: PCT/EP2022/084363

§ 371 Date: May 30, 2024

(30) Foreign Application Priority Data
Dec. 29, 2021 (WO) PCT/CN2021/142278
Jan. 26, 2022 (EP) 22153355.7

(51) Int. Cl. A61K 8/25; A61K 8/02; A61Q 17/00; A61Q 19/00

(52) U.S. Cl. CPC A61K 8/25; A61K 8/0279; A61Q 17/00; A61Q 19/00; A61K 2800/412; A61K 2800/651

(57) ABSTRACT

Disclosed is a personal care composition comprising porous silica having an average diameter of 2 to 100 microns and lignin compound.

### FIELD OF THE INVENTION

The present invention relates to a personal care composition. In particular, the present invention is related to a personal care composition comprising porous silica and lignin compound for preventing pollutant.

### BACKGROUND OF THE INVENTION

Air pollution is a big problem, particularly in some of the developing countries. Particulate matter (PM) is one of the important to affect the air quality which are inhalable particles composed of sulphate, nitrates, ammonia, sodium chloride, black carbon, mineral dust and water. Particles with a diameter of less than 10 microns (PM10), including fine particles less than 2.5 microns (PM2.5) pose the greatest risks to health, as they can enter the lungs and the bloodstream. In addition, PM may bring adverse effects of pollution on human skin. These adverse effects include premature ageing, development of fine lines and wrinkles, pigmented spots, hyperpigmentation, rash and inflammation.

Therefore, we have recognized there is a need to develop a person care composition which is able to protect the skin from harmful effects of atmospheric pollutants. The present inventors developed a personal care composition comprising porous silica having an average diameter of 2 to 100 microns and lignin compound. It was surprisingly found that such composition is capable of significantly reducing the deposition of pollutant or blocking the pollutant after such composition is applied.

### SUMMARY OF THE INVENTION

In a first aspect, the present invention is directed to a personal care composition comprising porous silica having an average diameter of 2 to 100 microns and lignin compound.

In a second aspect, the present invention is directed to use of a composition of the present invention for preventing pollutant, in particular particulate pollutant, from skin of an individual.

In a third aspect, the present invention is directed to a method of preventing pollutant from skin of an individual, comprising a step of applying the composition of the present invention to the skin.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

“Specific surface area” as used herein refers to specific surface area determined according to Brunauer-Emmett-Teller method. The value of the specific surface area may be measured by following ASTM standard D 3663-78.

“Diameter” as used herein refers to particle diameter in non-aggregated state unless otherwise stated. For polydisperse samples having particulate with diameter no greater than 1 μm, diameter means the z-average diameter measured, for example, using dynamic light scattering (see international standard ISO 13321) with an instrument such as a Zetasizer Nano™ (Malvern Instruments Ltd, UK) unless otherwise stated. For polydisperse samples having particulate with diameter no less than 1 μm, diameter means the apparent volume median diameter (D50, also known as ×50 or sometimes d(0.5)) of the particles measurable for example, by laser diffraction using a system (such as a Mastersizer™ 2000 available from Malvern Instruments Ltd) meeting the requirements set out in ISO 13320 unless otherwise stated.

Porous silica as used herein refers to the silica having distributed voids throughout the entire volume of the particle. The voids can be individual or connected by small size openings, similar to pore openings that separate larger spaces.

Preferably, the porous silica is non-fumed silica. The porous silica may be unmodified porous silica, hydrophilically modified or hydrophobically modified porous silica. Preferably the porous silica is hydrophobically modified porous silica, hydrophobically modified porous silica as used herein refers to silica having an oil absorption value of greater than 10 g of oil/100 g of particle measured in same manner as described in ASTM Method D281-84. More preferably the porous silica comprises silica having INCI (International Nomenclature of Cosmetic Ingredients) Name of silica silylate.

The specific surface area of the porous silica is preferably higher than 100 m²/g, more preferably from 150 to 1500 m²/g, even more preferably from 200 to 1000 m²/g and most preferably from 500 to 900 m²/g.

The porous silica is capable for absorbing large amounts of oils. Preferably, the porous silica is a porous silica microsphere having an oil absorption value of higher than 50 g/100 g, more preferably higher than 100 g/100 g and even more preferably higher than 120 g/100 g. The oil absorption value refers to the values measured in conformity with ASTM Method D281-84.

The porous silica preferably has an average diameter of 2 to 70 microns, more preferably from 3 to 40 microns, even more preferably from 4 to 20 microns, and most preferably from 5 to 15 microns. To have a better sensory, the porous silica is preferably substantially uniform in size which means less than 5% of the porous silica have a diameter less than 0.5 times the average diameter and less than 5% of the porous silica have a diameter greater than 1.5 times the average diameter. In another aspect, the range of the diameter of the porous silica is preferably 0.8 to 1.2 times the average diameter, more preferably 0.9 to 1.1 times the average diameter.

The porous silica is preferably present in amount of 0.01 to 20% by weight of the composition, more preferably from 0.05 to 14%, even more preferably from 0.2 to 9%, and most preferably from 0.5 to 5% by weight of the composition.

“Lignin compound” as used herein refers to a compound comprising lignin or a modified lignin. Typically the modified lignin means lignin which has been modified through alkylation, alkoxylation, sulfonation, sulfation, alkoxy sulfation, sulfomethylation and combinations thereof. Preferably, the lignin compound comprises lignin, lignosulfonate, cationically-modified lignin, amino lignin, alkylated lignin, crosslinked lignosulfonate, crosslinked lignin or a combination thereof. More preferably the lignin compound comprises lignin, lignosulfonate, or a combination thereof. Even more preferably the lignin compound comprises lignosulfonate. Still even more preferably the lignin compound is lignosulfonate.

Lignosulphonate refers to sulphonated lignin. The lignosulphonate may be provided in the form of a salt with a suitable cation, for example sodium, magnesium, ammonium, calcium or a combination thereof. More preferably the lignosulphonate is sodium lignosulphonate, magnesium lignosulphonate or a mixture thereof and most preferably the lignosulphonate is sodium lignosulphonate. Sodium lignosulphonate is exchangeable with sodium lignin sulfonate. Preferably the lignin compound comprises sodium lignosulfonate and most preferably the lignin compound is sodium lignosulfonate.

Preferably, the lignin compound has a weight-average molecular weight of from 300 to 1,000,000, and more preferably from 500 to 500,000. Preferably, the lignosulphonate has a weight-average molecular weight of from 300 to 1,000,000, and more preferably from 500 to 500,000.

Preferably, the lignin compound is present in amount of 0.01 to 20% by weight of the composition, more preferably 0.1 to 18%, even more preferably 0.5 to 15%, still even more preferably 1 to 10% and most preferably 2 to 8% by weight of the composition. Preferably, the lignosulphonate is present in amount of 0.01 to 20% by weight of the composition, more preferably 0.1 to 18%, even more preferably 0.5 to 15%, still even more preferably 1 to 10% and most preferably 2 to 8% by weight of the composition.

Preferably, the weight ratio of the lignin compound to the porous silica is 1:10 to 200:1, more preferably 1:3 to 70:1, even more preferably 1:1 to 24:1, and still even more preferably 2:1 to 12:1. Preferably, the weight ratio of the lignosulphonate to the porous silica is 1:10 to 200:1, more preferably 1:3 to 70:1, even more preferably 1:1 to 24:1, and still even more preferably 2:1 to 12:1.

The composition may comprise water in amount of 35 to 95% by weight of the composition, more preferably from 45 to 92%, even more preferably from 50 to 90%, most preferably from 68% to 88% by weight of the composition. The composition is preferably an emulsion and more preferably an oil-in-water emulsion.

The composition may comprise resorcinol derivative. Resorcinol derivative preferably refers to that at least one hydrogen on the ring structure and/or on a hydroxy group of the resorcinol replaced with an alkyl group, phenyl alkyl group. Preferably, the resorcinol derivative is 4-substituted resorcinol. Preferably, the resorcinol derivative is selected from 4-ethyl resorcinol, 4-butyl resorcinol, 4-hexyl resorcinol, phenylethyl resorcinol, or a mixture thereof, and more preferably, the resorcinol derivative comprises 4-hexyl resorcinol. The amount of the resorcinol derivative is preferably in the range of 0.00001 to 10%, more preferably from 0.001 to 5% and most preferably from 0.1 to 0.6% by weight of the total amount of the composition.

Preferably, the composition comprises Vitamin B3 compounds (including derivatives of vitamin B3). The vitamin B3 compounds comprises niacin, nicotinic acid, niacinamide or a mixture thereof. The most preferred vitamin B3 compound is niacinamide. Amount of Vitamin B3 compounds may be 0.1 to 10%, preferably 0.5 to 5% by weight of the composition.

The composition preferably additionally comprises one or more organic sunscreens. A wide variety of organic sunscreen is suitable for use in combination with the essential ingredients of this invention. Suitable UV-A/UV-B sunscreen include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof. A safe and effective amount of organic sunscreen may be used in the compositions useful in the subject invention. The composition preferably comprises from 0.1% to 10%, more preferably from 0.1% to 5%, of organic sunscreen by weight of the composition.

Preferably, the composition comprises polyhydric alcohol. Polyhydric alcohols may be selected from group of glycerin, propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol or a mixture thereof. Most preferred polyhydric alcohol is glycerol known also as glycerin. The amount of polyhydric alcohol may range anywhere from 0.1 to 20%, preferably 0.5 to 15% and more preferably 2 and 10% by weight of the composition.

Water-insoluble skin benefit agents may also be formulated into the compositions as conditioners and moisturizers. Examples include silicone oils; and vegetable triglycerides such as sunflower seed and cottonseed oils.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

Preservatives can desirably be incorporated into the compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients. Preservatives are preferably employed in amounts ranging from 0.01% to 2% by weight of the composition.

A variety of other optional materials may be formulated into the compositions. These may include: antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; and colorants.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

Preferably, the composition has a viscosity of at least 10 mPa·s, more preferably in the range 30 to 10000 mPa·s, even more preferably 50 to 5000 mPa·s, and most preferably 100 to 2000 mPa·s, when measured at 20 degrees C. at a relatively high shear rate of about 20 s⁻¹. Preferably, the composition is in the form of fluid.

Preferably, the personal care composition is a skin care composition. The term “skin” as used herein includes the skin on the face, neck, chest, abdomen, back, arms, under arms, hands, and legs. Preferably “skin” means includes the skin on the face and under arms, more preferably skin means skin on the face other than lips and eyelids.

Preferably, preventing pollutant refers to reducing contact of skin with a pollutant. More preferably, preventing pollutant comprising reducing deposition of pollutant on composition applied onto the surface and/or blocking pollutant in the composition applied onto the surface. Preferably, pollutant is particulate matter, more preferably PM2.5 and/or PM10.

The following examples are provided to facilitate an understanding of the invention. The examples are not intended to limit the scope of the claims.

### EXAMPLES

### Materials

| | | | | Average | Surface |
|---|---|---|---|---|---|
| | | INCI | | diameter | area |
| Supplier | Trade name | name | Morphology | (μm) | (m²/g) |
| Dow Chemical | DOWSIL ™ VM- | Silica | Porous | 5-15 | 600-800 |
| | 2270 | Silylate | | | |
| Solvay | TIXOSIL ® 68 | Silica | Porous | 200-300 | — |
| Guangdong Qichen New | Q701 | Silica | Porous | 0.98 | — |
| material Technology | | | | | |
| Co. Ltd. | | | | | |
| KOBO | MSS-500/3N | Silica | Non-porous | 5.5 | — |
| Tokyo Chemical Industry | | Sodium | — | — | — |
| Co. Ltd | | lignin | | | |
| | | sulfonate | | | |

### Example 1

This example demonstrates the effect of porosity of silica on the performance of pollutant deposition.

TABLE 1

Sample (wt %)*

| Ingredient | A | 1 |
|---|---|---|
| Water | To 100 | To 100 |
| MSS-500/3N | 1.00 | — |
| DOWSIL ™ VM-2270 | — | 1.00 |
| Sodium Lignin Sulfonate | 5.00 | 5.00 |
| Glycerin | 2.50 | 2.50 |
| Disodium EDTA | 0.10 | 0.10 |
| Carbopol Ultrez 10 | 0.40 | 0.40 |
| KOH | 0.54 | 0.54 |
| Xanthan Gum | 0.30 | 0.30 |
| NaOH | 0.22 | 0.22 |
| Phenylbenzimidazole Sulfonic Acid | 1.50 | 1.50 |
| Parsol 1789 | 1.00 | 1.00 |
| Octocrylene | 1.00 | 1.00 |
| Ethylhexyl Salicylate | 4.00 | 4.00 |
| Dimethicone 50 cSt | 1.00 | 1.00 |
| Glycol Stearate & Stearamide AMP | 1.39 | 1.39 |
| PEG-100 Stearate | 1.19 | 1.19 |
| Cetyl Alcohol | 0.37 | 0.37 |
| Glyceryl Stearate | 0.65 | 0.65 |
| Stearic Acid | 2.35 | 2.35 |
| BHT | 0.05 | 0.05 |
| Petrolatum | 0.01 | 0.01 |
| Phenoxyethanol | 0.40 | 0.40 |
| Niacinamide | 0.10 | 0.10 |
| Caprylyl Glycol | 0.50 | 0.50 |

The personal care compositions in Table 1 were formulated by following standard procedure.

The prepared sample A and 1 were applied onto six identical glass plates (22 mm×22 mm) evenly with a same amount and dried overnight. Then coated glass plates were placed into a home-made pollution simulation chamber with a temperature of 25° C. and relative humidity of 50%. Vehicle exhausts, as simulation of particulate matters, were supplied into the closed chamber at level of 400 μg/m³ for 8 hours. Then, the coated glass plates were kept in the closed chamber overnight. The amount of PAH (Polycyclic Aromatic Hydrocarbons) on coated glass plates were determined by GC-MS (Thermo scientific ISQ 7000 Single Quadrupole Mass Spectrometer and Thermo scientific Trace 1300 Gas Chromatograph) and summarized in Table 2.

TABLE 2

| | | Total amount of PAH deposition | |
|---|---|---|---|
| Sample | Type of silica | (ng) | P value |
| A | Non-porous silica | 338.0 | — |
| 1 | Porous silica | 243.8 | 0.0003^{#} |

^{#}Sample 1 vs. Sample A

As shown in Table 2, it was surprisingly found that the personal care composition of the present invention (Sample 1) provided a significant better performance for reducing PAH deposition than composition containing a non-porous silica (Sample A).

### Example 2

This example demonstrates the effect of diameter of the particles on the performance of pollutant deposition.

TABLE 3

| | Sample (wt %)* | | | | | |
|---|---|---|---|---|---|---|
| Ingredient | 1 | B | C | C′ | D | D′ |
| Water | To | To | To | To | To | To |
| | 100 | 100 | 100 | 100 | 100 | 100 |
| DOWSIL ™ | 3.00 | 3.00 | — | — | — | — |
| VM-2270 | | | | | | |
| TIXOSIL ® 68 | — | — | 3.00 | 3.00 | — | — |
| Q701 | — | — | — | — | 3.00 | 3.00 |
| Sodium Lignin | 5.00 | — | 5.00 | — | 5.00 | — |
| Sulfonate | | | | | | |
| Glycerin | 2.50 | 2.50 | 2.50 | 2.50 | 2.50 | 2.50 |
| Disodium EDTA | 0.10 | 0.10 | 0.10 | 0.10 | 0.10 | 0.10 |
| Carbopol Ultrez 10 | 0.40 | 0.40 | 0.40 | 0.40 | 0.40 | 0.40 |
| KOH | 0.54 | 0.54 | 0.54 | 0.54 | 0.54 | 0.54 |
| Xanthan Gum | 0.30 | 0.30 | 0.30 | 0.30 | 0.30 | 0.30 |
| NaOH | 0.22 | 0.22 | 0.22 | 0.22 | 0.22 | 0.22 |
| Phenylbenzimidazole | 1.50 | 1.50 | 1.50 | 1.50 | 1.50 | 1.50 |
| Sulfonic Acid | | | | | | |
| Parsol 1789 | 1.00 | 1.00 | 1.00 | 1.00 | 1.00 | 1.00 |
| Octocrylene | 1.00 | 1.00 | 1.00 | 1.00 | 1.00 | 1.00 |
| Ethylhexyl Salicylate | 4.00 | 4.00 | 4.00 | 4.00 | 4.00 | 4.00 |
| Dimethicone 50 cSt | 1.00 | 1.00 | 1.00 | 1.00 | 1.00 | 1.00 |
| Glycol Stearate & | 1.39 | 1.39 | 1.39 | 1.39 | 1.39 | 1.39 |
| Stearamide AMP | | | | | | |
| PEG-100 Stearate | 1.19 | 1.19 | 1.19 | 1.19 | 1.19 | 1.19 |
| Cetyl Alcohol | 0.37 | 0.37 | 0.37 | 0.37 | 0.37 | 0.37 |
| Glyceryl Stearate | 0.65 | 0.65 | 0.65 | 0.65 | 0.65 | 0.65 |
| Stearic Acid | 2.35 | 2.35 | 2.35 | 2.35 | 2.35 | 2.35 |
| BHT | 0.05 | 0.05 | 0.05 | 0.05 | 0.05 | 0.05 |
| Petrolatum | 0.01 | 0.01 | 0.01 | 0.01 | 0.01 | 0.01 |
| Phenoxyethanol | 0.40 | 0.40 | 0.40 | 0.40 | 0.40 | 0.40 |
| Niacinamide | 0.10 | 0.10 | 0.10 | 0.10 | 0.10 | 0.10 |
| Caprylyl Glycol | 0.50 | 0.50 | 0.50 | 0.50 | 0.50 | 0.50 |

*The level in this table refers the level of left-listed ingredients, unless otherwise specified.

The personal care compositions in Table 3 were formulated by following standard procedure.

The samples in Table 3 were coated and exposed to vehicle exhausts by following same procedure as described in Example 1. Then, the coated samples (a thin film on glass plate) with deposited pollution were peeled off by new tapes for five (5) times. Each tape was washed by acetone and the amounts of PAH and octocrylene (as marker of depth) on each tape were determined by GC-MS (Thermo scientific ISQ 7000 Single Quadrupole Mass Spectrometer and Thermo scientific Trace 1300 Gas Chromatograph).

Under the assumption that the concentration of octocrylene is consistent along the height of the film, the amount of octocrylene represents the height of the film formed by samples. A fitted equation for each sample was generated by plotting the data of accumulative PAH percentage and the height of the film. Then, the percentage of PAH blocked at the top 40% height of coated sample film based on the total PAH blocked by the sample film for each sample was obtained from the fitted equation. The improvements by the inclusion of lignin compound for sample 1, C and D were calculated by subtracting the percentage for sample having no lignin compound (Sample B, C′ and D′ respectively) from the percentage for the corresponding sample.

The results were summarized in Table 4.

TABLE 4

| Sample | Average diameter of silica (μm) | Improvement (%) |
|---|---|---|
| 1 | 5-15 | 26 |
| C | 200-300 | 17 |
| D | 0.98 | 18 |

As evidenced in Table 4, it was surprisingly found that the inclusion of the lignin compound is capable of improving the PAH block efficacy at the top 40% height of coated sample film of personal care composition containing porous silica having average diameter within the scope of present invention (Sample 1) than composition porous silica having average diameter outside of the present invention (Sample C or D).

1. A personal care composition comprising porous silica having an average diameter of 2 to 100 microns and lignin compound.

2. The personal care composition according to claim 1, wherein the porous silica is hydrophobically-modified porous silica, wherein the porous silica comprises silica silylate.

3. The personal care composition according to claim 1, wherein the porous silica has a specific surface area higher than 100 m²/g.

4. The personal care composition according to claim 1, wherein the porous silica has an average diameter of 2 to 70 microns.

5. The personal care composition according to claim 1, wherein the porous silica is present in an amount of 0.01 to 20% by weight of the composition.

6. The personal care composition according to claim 1, wherein the lignin compound comprises lignosulfonate.

7. The personal care composition according to claim 1, wherein the lignin compound is present in an amount of 0.01 to 20% by weight of the composition.

8. The personal care composition according to claim 1, wherein the lignin compound to the porous silica is present in a weight ratio of 1:10 to 200:1.

9. The personal care composition according to claim 1, wherein the composition comprises water in an amount of 45 to 92% by weight of the composition.

10. The personal care composition according to claim 1, wherein the composition is an oil-in-water emulsion.

11. (canceled)

12. A method of preventing pollutant from skin of an individual, comprising a step of applying the personal care composition according to claim 1, to the skin.

13. The personal care composition according to claim 1, wherein the porous silica has a specific surface area of 200 to 1000 m²/g.

14. The personal care composition according to claim 1, wherein the porous silica has a specific surface area of 500 to 900 m²/g.

15. The personal care composition according to claim 1, wherein the porous silica has an average diameter of 4 to 20 microns.

16. The personal care composition according to claim 1, wherein the porous silica has an average diameter of 5 to 15 microns.

17. The personal care composition according to claim 1, wherein the porous silica is present in an amount of 0.2 to 9% by weight of the composition.

18. The personal care composition according to claim 1, wherein the lignin compound is present in an amount of 0.5 to 15% by weight of the composition.

19. The personal care composition according to claim 1, wherein the lignin compound to the porous silica is present in a weight ratio of 1:1 to 24:1.

20. The personal care composition according to claim 1, wherein the lignin compound to the porous silica is present in a weight ratio of 2:1 to 12:1.

21. The personal care composition according to claim 1, wherein the lignin compound comprises sodium lignosulfonate.
